(19) United States
(12) Patent Application Publication
(10) Pub. No.: US 2014/0343399 A1
(43) Pub. Date: Nov. 20, 2014

(54) Seed-Based Connectivity Analysis in Functional MRI

(71) Applicant: Stefan Posse, Albuquerque, NM (US)

(72) Inventor: Stefan Posse, Albuquerque, NM (US)

(21) Appl. No.: 14/281,630

(22) Filed: May 19, 2014

Related U.S. Application Data
(60) Provisional application No. 61/825,192, filed on May 20, 2013.

(51) Int. Cl. A61B 5/055; A61B 5/00

(52) U.S. Cl. CPC A61B 5/055; A61B 5/7203; A61B 5/4064
USPC 600/410

(57) ABSTRACT

Functional MRI (fMRI) methods are presented for utilizing a magnetic resonance tomograph to map connectivity between brain areas in the resting state in real-time without the use of regression of confounding signal changes. They encompass: (a) iterative computation of the sliding window correlation between the signal time courses in a seed region and each voxel of an fMRI image series, (b) Fisher Z-transformation of each correlation map, (c) computation of a running mean and a running standard deviation of the Z-maps across a second sliding window to produce a series of meta mean maps and a series of meta standard deviation maps, and (d) thresholding of the meta maps. This methodology can be combined with regression of confounding signals within the sliding window. It is also applicable to task-based real-time fMRI, if the location of at least one task-activated voxel is known.

### REFERENCE TO RELATED APPLICATIONS

Applicant claims priority of U.S. Provisional Application No. 61/825,192, filed on May 20, 2013 for SYSTEM AND METHODS FOR SEED-BASED CONNECTIVITY ANALYSIS IN FUNCTIONAL MAGNETIC RESONANCE IMAGING of Stefan Posse, Inventor.

### FEDERALLY SPONSORED RESEARCH

The present invention was not made with government support. As a result, the Government has no rights in this invention.

### BACKGROUND OF THE INVENTION

1. Technical Field of the Invention

This invention relates to functional magnetic resonance imaging (fMRI) and more specifically to improved fMRI system and methods for mapping the temporal dynamics of connectivity in resting state fMRI in real-time with increased tolerance to movement, respiration and other sources of confounding signal changes. The invention is also suitable for mapping functional networks and their connectivity in task-based fMRI.

2. Description of the Prior Art

Resting State Functional MRI

Functional connectomics using resting state fMRI is a rapidly expanding task-free approach, which is expected to have significant clinical impact¹⁻⁵. Mapping of intrinsic signal variation mostly in the low frequency band less than 0.1 Hz has emerged as a powerful tool and adjunct to task-related fMRI and fiber tracking based in diffusion tensor imaging (DTI) for mapping functional connectivity within and between resting state networks (RSNs)^{6-10.} Recent studies have shown that dozens of different RSNs can be measured across groups of subjects¹¹⁻¹². Anti-correlations between the default mode network and task-positive networks provide insights into competitive mechanisms that control resting state fluctuations⁸⁻¹³. There is increasing evidence that RSNs are not stationary^{14, 15} and that correlations with fluctuations in other measurements, such as a-power in EEG¹⁶ and transient (˜100 ms) topographies of EEG current source densities (microstates)¹⁷⁻¹⁹ exist. Variations in ongoing activity have been shown to predict changes in task performance and alertness, highlighting their importance for understanding the connection between brain activity and behavior^{20,21}. Resting state correlation mapping has been shown to be a promising tool for reliable functional localization of eloquent cortex in healthy controls, and patients with brain tumors and epilepsy¹⁻⁴. It has been suggested that this task-free paradigm may provide a powerful approach to map functional anatomy in patients without task compliance, which allows multiple brain systems to be determined in a single scanning session¹. Recent studies have investigated non-stationarity, which is prominent in the resting-state, and demonstrated dynamic changes in network connectivity²²⁻²⁴. There is now emerging evidence that these fluctuations differ in clinical populations compared to healthy controls. There is also considerable interest in characterizing resting state connectivity at much higher frequencies (up to 5 Hz) than detectable with traditional resting state fMRI²⁵⁻²⁷.

Seed-based correlation analysis²⁸ and spatial independent component analysis (ICA)²⁹ are the principal tools to map functional connectivity, which have been shown to provide similar results²⁸⁻³⁰. ICA performs spatial filtering, which enables segregation of spatially overlapping components. However, source separation with ICA is sensitive to the selection of the model order, which is a priori unknown and necessitates dimensionality estimation approaches, such as the minimum description length (MDL), Bayesian information criterion (BIC) and Akaike's information criterion (AIC)^{29,31}. Furthermore, automated ordering of ICA components to enable consistent identification of resting state networks is not yet feasible. Application of ICA in individual subject data to separate signal sources of resting state connectivity is severely constrained by the low contrast-to-noise-ratio of resting state signal fluctuations, as well as aliasing of cardiac- and respiration-related signal fluctuations, which limits clinical applications. Adaptation of ICA for real-time fMRI is only at the feasibility stage³²⁻³⁴.

Seed-based connectivity analysis (SBCA) provides high sensitivity for mapping RSN connectivity and enables straightforward interpretation of RSN connectivity in single subjects, which makes this approach attractive for clinical applications²⁸⁻³⁰. Seed-based connectivity measures have been shown to be the sum of ICA-derived within- and between-network connectivities³⁵. However, SBCA is highly sensitive to confounding signal sources from structured noise (signals of no interest) that requires regression with possible loss of RSN information and from other overlapping RSNs that are segregated in ICA³⁵. Furthermore, it suffers from variability inherent in investigator-specific and subject-specific seed selection³⁶. Regression of confounding signals, which typically includes the average signal from up to three brain regions (whole brain over a fixed region in atlas space, ventricles, and white matter in the centrum semiovale) is an empirical approach that is widely used, but it lacks a rigorous experimental validation. Furthermore, the regression of the global mean signal remains highly controversial. Movement during the fMRI acquisition is a major confound for resting state connectivity studies obscuring networks as well as creating false-positive connections^{37,38} despite state-of-the-art motion “correction” in post-processing. The high sensitivity of resting state fMRI to head motion additionally requires regression of movement parameters, which typically includes the six parameters of motion correction and their derivatives. Detrending of these signals using regression is computationally intensive and may remove RSN signal changes that are temporally correlated with confounding signals. A seed-based approach that does not require regression of confounding signal changes is thus highly desirable, but not yet described in the prior art.

Adaptation of SBCA for real-time fMRI is of considerable interest. Monitoring of resting state connectivity dynamics in real-time enables assessment of data quality, movement artifacts and the sensitivity and specificity of detecting resting state connectivity. Real-time monitoring of these dynamics is not only expected to improve consistency of data quality in clinical research studies, but will also contribute to our understanding of the neurophysiological mechanisms underlying the resting state dynamics that are currently insufficiently understood. None of the prior art describes a resting state fMRI analysis method to compute resting state connectivity in real-time, which requires updating statistical resting state connectivity maps on a TR-by-TR basis.

High-Speed fMRI

The measurement of functional connectivity in the resting state has been limited, in part, by sensitivity and specificity constraints of current fMRI data acquisition methods that predominantly rely on blood oxygenation level dependent contrast (BOLD) to detect brain activity. Conventional fMRI methods are acquired with a TR of 2-3 s. Echo planar imaging (EPI) methods necessitate long scan times and detection of resting state signal fluctuation suffers from temporally aliased physiological signal fluctuation, despite ongoing efforts to develop post-acquisition correction methods³⁹⁻⁴². Recent advances in high-speed fMRI method development that enable un-aliased sampling of physiological signal fluctuation have considerably increased sensitivity for mapping task-based activation and functional connectivity, as well as for detecting dynamic changes in connectivity over time⁴³⁻⁴⁵. Using ultra-high speed fMRI methods the TR can be as short as 50 ms. High temporal resolution fMRI thus improves separation of resting state networks using data driven analysis approaches⁴³ and may facilitate detecting the temporal dynamics of resting state networks at much higher frequencies (up to 5 Hz) than detectable with traditional resting state fMRI^{25-27,46}. Seed-based resting state data analysis approaches that take advantage of the fast encoding speed of high-speed fMRI are needed to realize the full potential and sensitivity of resting state fMRI in single subjects.

Task-Based fMRI:

Task-based fMRI is typically performed using a model-based analysis that uses the time course of task activation and a convolution with a hemodynamic response model to detect brain areas with task-related activation. The most frequently used methods to detect brain activation employ correlation analysis or the general linear model. However, the sensitivity for detecting brain activation critically depends on the accuracy of the model to fit the measured data, which may be confounded by time delays in task execution, changes in task performance over time that affect the amplitude and the time course of the task-related BOLD fMRI signal changes and regional differences in the hemodynamic response shape and amplitude. Accurate modeling of the signal response is particularly critical in event-related fMRI. Movement is a major confound in task-based fMRI that can obscure task-related activation and create false-positive activation. Task-related fMRI is also sensitivity to signal drift and physiological signal pulsation. It is desirable to develop data driven analysis methods to detect task-based brain activation without the need of priori time course modeling. In many experimental situations a brain area involved in task activation is known or can be found using a model-based analysis. A data driven analysis, which samples much finer detail of the signal response and which is feasible in case prior information about the localization of the activation is available, may provide much greater sensitivity than model-based approach. None of the prior art describes a seed-based approach to map task-based activation that does not require regression of confounding signal changes.

A real-time correlation analysis of task-based fMRI is known from Cox, R. W., Jesemanovicz, A., Hyde, J. S., Mag. Reson. Med. 1995, 33, 230, which supports the suppression of low-frequency noise by means of a detrending procedure. This method performs a cumulative correlation analysis for task-based fMRI. However, this method is not applicable to measuring resting state connectivity in fMRI data. U.S. Pat. No. 6,388,443, which is prior art by the inventor, discloses a method of sliding window correlation analysis with detrending of low frequency noise for applications in task-based real-time fMRI. However, this method is not applicable to measuring resting state connectivity in fMRI data.

### SUMMARY OF THE INVENTION

Embodiments of the invention provide systems and methods for utilizing a magnetic resonance tomograph to map resting state connectivity between brain areas in real-time during the ongoing scan without using regression of confounding signal changes (signals of no interest). An exemplary method encompasses the steps of (a) measuring a functional MRI (fMRI) image series, (b) extraction of the signal time course in a seed region, (c) iterative computation of the sliding window correlation between the signal time courses in the seed region and a in a plurality of voxels in a target region of the fMRI image series using a short sliding window width that is consistent with the Nyquist sampling rate of the resting state signal spectrum, (d) Fisher Z-transformation of each correlation map to generate Z-maps, (e) computation of meta-statistics using a running mean and a running standard deviation of the Z-maps across a second sliding window to produce a series of sliding window meta mean maps and sliding window meta standard deviation maps, and (e) thresholding of each meta mean map and each meta standard deviation map using either the meta map itself or the meta standard deviation map itself or a combination thereof. Extensions of this invention encompass additionally the steps of iterative sliding window correlation analysis with the computation of a weighted running mean and a weighted running standard deviation of the Z-maps using weights that decrease with increasing level of signals of no interest within the first sliding window, and the computation of cumulative meta-statistics. This approach can be further extended using detrending of confounding signals within the first sliding window. This invention is also applicable to mapping brain function during task execution in real time, if the location of at least one task-activated voxel is known to measure a seed signal time course.

It is an object of the present invention to improve the sensitivity and the specificity of detecting resting state connectivity in the brain using seed based correlation analysis of fMRI signal changes in different brain regions, without the need for regression of confounding signal sources, and to enable the detection of dynamic changes in resting state connectivity during an ongoing real-time fMRI scan.

It is another object of the present invention to improve the sensitivity and specificity of detecting activation and connectivity in task-related functional networks without using a hemodynamic response model and without the need for regression of confounding signal sources, and to enable the detection of dynamic changes in activation and connectivity in task-related functional networks during an ongoing real-time fMRI scan.

It is still another object of the present invention to combine this approach with detrending of confounding signal sources as part of the sliding window correlation analysis to further improve the sensitivity and the specificity of detecting resting state connectivity and task-related functional networks in the brain during an ongoing real-time fMRI scan.

It is still another object of the present invention to apply this approach to other signal acquisition systems (e.g. magnetic resonance imaging (MRI) and spectroscopy (MRS), parallel MRI using array RF coils, electroencephalography, magneto-encephalography, optical imaging, recordings from electrode arrays, phased array radar, and radio-telescope arrays), where correlation between signals from different signal sources is examined in the presence of confounding signals of no interest.

These and significant other advantages of the present invention will become clear to those skilled in this art by careful study of this description, accompanying drawings and claims.

### BRIEF DESCRIPTION OF THE DRAWINGS

The preferred embodiments of the invention will be described in conjunction with the appended drawings provided to illustrate and not to limit the invention, where like designations denote like elements, and in which:

FIG. 1 shows the analysis steps to compute a cumulative seed-based meta-statistics image. The fMRI image data are preprocessed using standard methods, such as motion correction, slice time correction, spatial normalization, spatial smoothing and temporal low pass filtering. Seed and confound signal time courses are extracted to perform a seed-based sliding window correlation and computation of weights based on the level of confounding signal changes with each sliding window position. Weighted cumulative meta statistics are performed across the correlation maps and thresholds are applied to the resulting meta maps.

FIG. 2 shows the BEST MODE of the invention, which computes a sliding window seed-based meta-statistics image. The fMRI image data are preprocessed using standard methods, such as motion correction, slice time correction, spatial normalization, spatial smoothing and temporal low pass filtering. Seed and confound signal time courses are extracted to perform a seed-based sliding window correlation and computation of weights based on the level of confounding signal changes with each sliding window position. Weighted meta-statistics are performed across the correlation maps within a second sliding window and thresholds are applied to the resulting meta maps.

FIG. 3 shows on the left (FIGS. 3a-d) a simulation of two signal time courses, which contain a common signal oscillation to simulate a resting state network (RSN), random noise and different confounding signals. The corresponding Z-scores obtained by averaging data segments with different sliding window widths are shown on the right (FIGS. 3e-h). (a) Signal time courses without confounding signals. The embedded signal oscillation of the RSN is shown with on offset. (b) Signal time courses with identical spikes in both signals. (c) Signal time courses with identical signal drift in both signals. (d) Signal time courses with identical signal offset in both signals.

FIG. 4 shows on the left a simulation of two signal time courses, which contain a common signal oscillation to simulate a resting state network (RSN), random noise and different confounding signals (FIGS. 4a-d). The corresponding Z-scores obtained by averaging data segments with different sliding window widths are shown on the right (FIGS. 4e-h). (a) Signal time courses without confounding signals. The embedded signal oscillation of the RSN is shown with on offset. (b) Signal time courses with a spike in one of the signals. (c) Signal time courses with a signal drift in one of the signals. (d) Signal time courses with a signal offset in one of the signals.

FIG. 5 shows on the left a simulation of two signal time courses that consist of random noise and different confounding signals (FIGS. 5a-d). The corresponding Z-scores obtained by averaging data segments with different sliding window widths are shown on the right (FIGS. 5e-h). (a) Signal time courses without confounding signals. (b) Signal time courses with identical spike in both signals. (c) Signal time courses with identical signal drift in both signals. (d) Signal time courses with identical signal offset in both signals.

FIG. 6 shows seed-based connectivity analyses of ultra-high speed resting state fMRI data in vivo in the human brain. The data were measured using multi-slab echo volumar imaging with 136 ms temporal resolution and a 5 min scan time. (a) A correlation analysis across the entire 5 min scan without regression of confounding signal changes using a seed in the left the sensorimotor cortex results in widespread artifacts with high signal correlation across all slices (arrows) and head movement related artifacts at the edges (arrows). (b) A sliding-window correlation analysis of the same data using a 4 s sliding window, the same seed region and a cumulative running meta-mean statistics across the series of sliding window correlation maps removes the artifacts. The resulting meta mean map displays low correlation across all slices with the exception of high correlation in the sensorimotor network (arrows). No regression of confounding signals was applied. (c-f) Seed-based sliding window correlation analysis with a running meta mean statistics and a seed in the left auditory cortex displays consistent detection of the auditory resting state network using sliding window widths of (c) 15 s, (d) 4 s, (e) 2 s and (f) 1 s. Note that no correlation threshold was applied in (a) and (b). The correlation values in (c) were threshold with correction for degrees of freedom as described in eq. 13 in ⁴⁷ using a cross-correlation threshold of 0.52.

FIG. 7 shows the arrangement of a conventional MRI apparatus, which is prior art.

### DETAILED DESCRIPTION OF THE PRESENT INVENTION

The invention employs seed-based correlation analysis across a short sliding window and a running mean and a running standard deviation across the dynamically updated Z-transformed correlation maps, which as our computer simulations and preliminary data show, is highly effective for detecting resting state signal fluctuations and for suppressing confounding signals of no interest in resting state fMRI without relying on regression of confounding signals. Movements and rapidly changing confounding signals, which create strong correlations in conventional seed-based resting state analysis, are strongly reduced using this meta-statistics approach. Performing this meta-statistics approach using a sliding window enables mapping of dynamic changes in connectivity during the scan. Moreover, the computational performance of the methodology considerably exceeds that of conventional seed-based analysis methods using regression of confounding signals. This approach is therefore suitable for real-time mapping of dynamic changes in resting state network connectivity using conventional EPI methods with repetition times of 1-3 s, as well as recently introduced ultra-high-speed functional MRI methods, such as Multi-Band EPI, Echo-Volumar Imaging (EVI), Magnetic Resonance Encephalography and inverse functional MRI with repetition times as short as 10 s of milliseconds. This approach is particularly well suited for the large degrees of freedom afforded by high-speed fMRI.

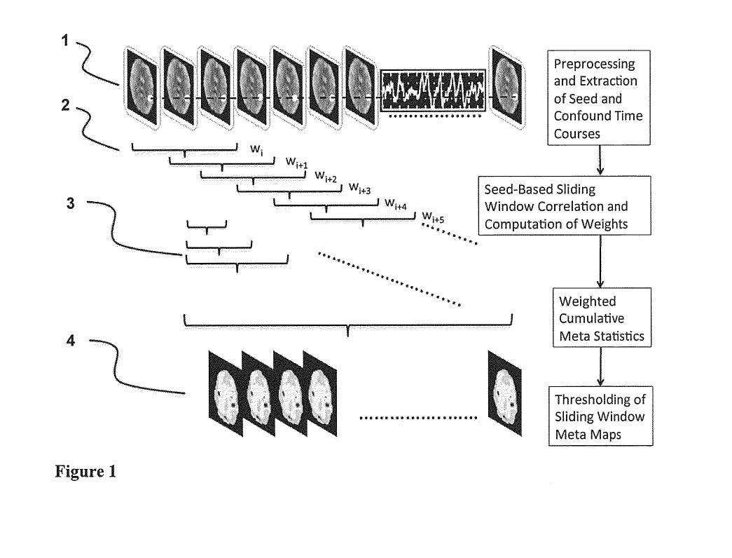

A preferred implementation of the method includes the following steps shown in FIG. 1:
1. Measure an fMRI image series I(r, tₙ) with voxel positions r at N discrete time points tₙ starting at t₁ and ending at t_{N} using a sampling interval Δt that is equal to or shorter than the Nyquist sampling interval 1/(2f) required for sampling a periodic resting state signal with frequency f that is present in a seed region and in a plurality of voxels outside of the seed region.
2. Apply standard preprocessing steps including motion correction, slice time correction, spatial normalization into the space of a standardized brain atlas, spatial smoothing and time domain low pass filtering 1.
3. Extract a data series S(P, tₙ) from I(r, tₙ) 1, which is the mean signal intensity of the seed region with one or more voxel positions P.
4. Perform an iterative computation of the sliding window correlation between the signal time courses in the seed region S(P, tₙ) and in a plurality of voxels with positions r of the fMRI image series I(r, tₙ) 2 using the sliding window correlation method described in⁴⁸ with sliding window width K<N where K>=1/(2fΔt), resulting in a series of correlation maps R_{K}(r, tₙ).
5. Compute the Fisher Z-transform Z(r,tₙ) of R_{K}(r,tₙ).
6. Compute M(r, tₙ) the cumulative meta-statistics across the series of Z-maps Z(r, tₙ), which includes, but is not limited to the running mean M(mean, r, tₙ) and the running standard deviation M(SD, r, tₙ) of Z(r, tₙ), and combinations thereof 3, preferably using the method described in⁴⁹.
7. Perform thresholding of the cumulative metastatistics map M(r, tₙ), the correlation maps R(r, tₙ) and the Z-maps Z(r,tₙ) using either of them 4. Examples include, but are not limited to:
   a. Map the running means M(mean, r, tₙ) that are either less or greater than a threshold.
   b. Map the running means M(mean, r, tₙ) whose running standard deviations M(SD, r, tₙ) is either less or greater than a threshold.
   c. Map the running standard deviations M(SD, r, tₙ) either less or greater than a threshold.
   d. Map the running standard deviations M(SD, r, tₙ) whose running mean M(mean, r, tₙ) is either less or greater than a threshold.
   e. Map the ratios of the running means M(mean, r, tₙ) over the running standard deviations M(SD, r, tₙ) whose correlation is either less or greater than a threshold.
   f. Map the ratios of the running standard deviations M(SD, r, tₙ) over the running means M(mean, r, tₙ) whose correlation is either less or greater than a threshold
   g. Map the correlation R(r, tₙ) or the Z-scores Z(r,tₙ) whose running mean M(mean, r, tₙ) is either less or greater than a threshold.

An alternative implementation also shown in FIG. 1 modifies the above step 6 as follows to improve the rejection of confounding signal changes:
6. Measure the time course C(j, tₙ) of confounds j in the data I(r, tₙ) measured within the sliding window K 1, including rigid body head movement parameters (3 translations and 3 rotations) and their temporal derivatives, artifact signals and signal fluctuation in selected regions of interest (e.g. white matter, CSF). Movement may be detected using standard motion correction algorithms implemented in widely available fMRI data analysis tools such as SPM (www.fil.ion.ucl.ac.uk/spm/) or using real-time motion correction as described in ⁵⁰.
7. Compute w(tₙ), a confidence metric of Z(r,tₙ), which decreases when increasing confounds are detected in the data measured within the sliding window K, and increases when these confounds diminish 2. A preferred implementation uses the 6 measured translation and rotation parameters Δr(t), their temporal derivatives and the temporal derivative of the signal δs(t) from a reference region (e.g. bilateral ROI in the centrum semiovale) to compute the weights w(t):

w(t)=1/(1+(α₁∫_{t-Δ}^{t}Δr(τ)dτ+α₂∫_{t-Δ}^{t}|δ(Δr(τ)/dτ|dτ+α₃∫_{t-Δ}^{t}|δs(τ)/dτ|dτ))
. The scale factors aᵢ are determined experimentally. Polynomial functions of the arguments of the integrals and thresholds for applying weights based on the movement parameters may be used to further refine the computation of the weights.
8. Compute M_{w}(r, tₙ) the weighted cumulative meta-statistics across the series of Z-maps

Z(r, tₙ), which includes the running mean M(mean, r, tₙ) and the running standard deviation M(SD, r, tₙ) of Z(r, tₙ)*w(tₙ), and combinations thereof 3, preferably using the method described in⁴⁹.

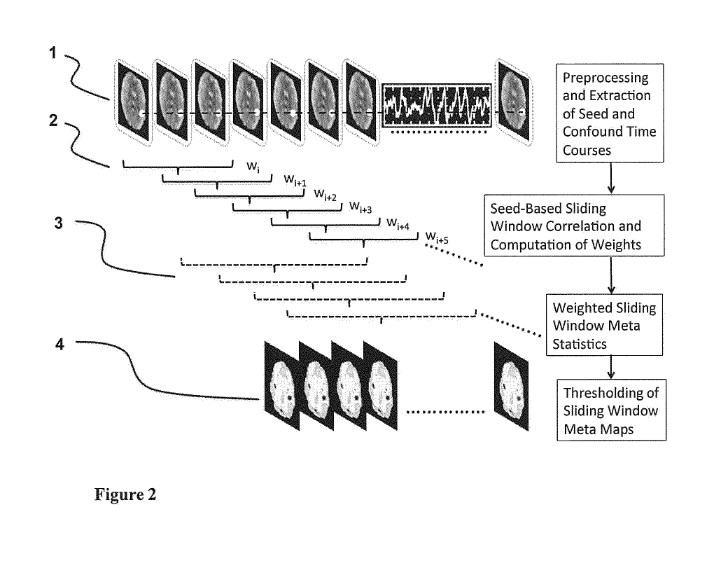

The Best Mode of the present invention is shown in FIG. 2. This mode includes the following steps:
1. Measure an fMRI image series I(r, t,₁) with voxel positions r at N discrete time points tₙ starting at t₁ and ending at t_{N} using a sampling interval Δt that is equal to or shorter than the Nyquist sampling interval 1/(2f) required for sampling a periodic resting state signal with frequency f that is present in a seed region and in a plurality of voxels outside of the seed region.
2. Apply standard preprocessing steps including motion correction, slice time correction, spatial normalization into the space of a standardized brain atlas, spatial smoothing and time domain low pass filtering 1.
3. Extract a data series S(P, tₙ) from I(r, tₙ) 1, which is the mean signal intensity of a seed region with one or more voxel positions P.
4. Perform an iterative computation of the sliding window correlation between the signal time courses in the seed region S(P, tₙ) and in a plurality of voxels with positions r of the fMRI image series I(r, tₙ) 2 using the sliding window correlation method described in⁴⁸ with sliding window width K<N, where K>=1/(2fΔt), resulting in a series of correlation maps R_{K}(r, tₙ).
5. Compute the Fisher Z-transform Z(r,tₙ) of R_{K}(r, tₙ).
6. Compute M(L, r, tₙ) the sliding window meta-statistics with sliding window width L<N across a range of recently computed Z-maps Z(r, tᵢ), where K+L−1 is the desired temporal resolution for monitoring changes in Z-scores and i=n−L, n−L+1, . . . , n−1, n. This sliding window meta-statistics includes, but is not limited to the running sliding window mean M(L, mean, r, tᵢ) and the running sliding window standard deviation M(L, SD, r, tᵢ) of Z(r, tᵢ), and combinations thereof 3, in which, with continuing data measurement, the respective oldest values are discarded and the newest data values are employed in the computation.
7. Perform thresholding of the sliding window metastatistics map M(L, r, tₙ), the correlation maps R(r, tₙ) and the Z-maps Z(r,tₙ) using either of them 4. Examples include, but are not limited to:
   a. Map the sliding window running means M(L, mean, r, tₙ) that are either less or greater than a threshold.
   b. Map the sliding window running means M(L, mean, r, tₙ) whose sliding window running standard deviations M(L, SD, r, tₙ) is either less or greater than a threshold.
   c. Map the sliding window running standard deviations M(L, SD, r, tₙ) either less or greater than a threshold.
   d. Map the sliding window running standard deviations M(L, SD, r, tₙ) whose sliding window running mean M(L, mean, r, tₙ) is either less or greater than a threshold.
   e. Map the ratios of the sliding window running means M(L, mean, r, tₙ) over the sliding window running standard deviations M(L, SD, r, tₙ) whose correlation is either less or greater than a threshold.
   f. Map the ratios of the sliding window running standard deviations M(L, SD, r, tₙ) over the sliding window running means M(L, mean, r, tₙ) whose correlation is either less or greater than a threshold
   g. Map the correlation R(r, tₙ) or the Z-scores Z(r, tₙ) whose sliding window running mean M(L, mean, r, tₙ) is either less or greater than a threshold.

An alternative implementation also shown in FIG. 2 modifies the above step 6 as follows to improve the rejection of confounding signal changes:
6. Measure the time course C(j, tₙ) of confounds j in the data I(r, tₙ) measured within the sliding window K 1, including rigid body head movement parameters (3 translations and 3 rotations) and their temporal derivatives, artifact signals and signal fluctuation in selected regions of interest (e.g. white matter, CSF). Movement may be detected using standard motion correction algorithms implemented in widely available fMRI data analysis tools such as SPM (www.fil.ion.ucl.ac.uk/spm/) or using real-time motion correction as described in⁵⁰.
7. Compute w(tₙ), a confidence metric of Z(r,tₙ), which decreases when increasing confounds are detected in the data measured within the sliding window K, and increases when these confounds diminish 2. A preferred implementation uses the 6 measured translation and rotation parameters Δr(t), their temporal derivatives and the temporal derivative of the signal from a bilateral ROI in the centrum semiovale δs(t) to compute the weights w(t):

w(t)=1/(1−(α₁∫_{t-Δ}^{t}Δr(τ)dτ+α₂∫_{t-Δ}^{t}|δ(Δr(τ)/dτ|dτ−α₃∫_{t-Δ}^{t}|δs(τ)/dτ|dτ)). The
scale factors aᵢ are determined experimentally. Polynomial functions of the arguments of the integrals and thresholds for applying weights based on the movement parameters may be used to further refine the computation of the weights.
8. Compute M_{w}(L, r, tₙ) the weighted sliding window meta-statistics with sliding window width L across a range of recently computed weighted Z-maps Z(r, tᵢ)*w(tᵢ), where K+L−1 is the desired temporal resolution for monitoring changes in Z-scores and i=n−L, n−L+1, . . . , n−1, n. The sliding window meta-statistics includes the running mean M(mean, r, tₙ) and the running standard deviation M(SD, r, tᵢ) of Z(r, tᵢ)*w(tᵢ), and combinations thereof 3, preferably using the method described in⁴⁹.

Yet another implementation of the method adds the following steps to the above sliding window meta-statistics approach:
9. Computation of the M_{c}(r, tₙ) the cumulative meta-statistics across the series of sliding window meta-statistics maps M(L, r, tₙ), which includes the running mean and the running standard deviation of M(L, r, tₙ), and combinations thereof, preferably using the method described in⁴⁹.
10. Perform thresholding of the cumulative metastatistics maps M_{c}(r, tn) and the sliding window meta-statistics maps M(r,tₙ) using either of them. Examples include, but are not limited to:
   a. Map the running means M(mean, r, tₙ) that are either less or greater than a threshold.
   b. Map the running means M(mean, r, tₙ) whose running standard deviations M(SD, r, tₙ) is either less or greater than a threshold.
   c. Map the running standard deviations M(SD, r, tₙ) either less or greater than a threshold.
   d. Map the running standard deviations M(SD, r, tₙ) whose running mean M(mean, r, tₙ) is either less or greater than a threshold.
   e. Map the ratios of the running means M(mean, r, tₙ) over the running standard deviations M(SD, r, t,ₙ) whose correlation is either less or greater than a threshold.
   f. Map the ratios of the running standard deviations M(SD, r, tₙ) over the running means M(mean, r, tₙ) whose correlation is either less or greater than a threshold
   g. Map the correlation R(r, tₙ) or the Z-scores Z(r,tₙ) whose running mean M(mean, r, tₙ) is either less or greater than a threshold.

Yet another implementation of this methodology adds detrending of confounding signal changes to the sliding window correlation analysis within the window width K in the above processing steps labeled 4 to further reduce the effect of confounding signal changes, preferably using the approach described in⁴⁸ and in U.S. Pat. No. 6,388,443.

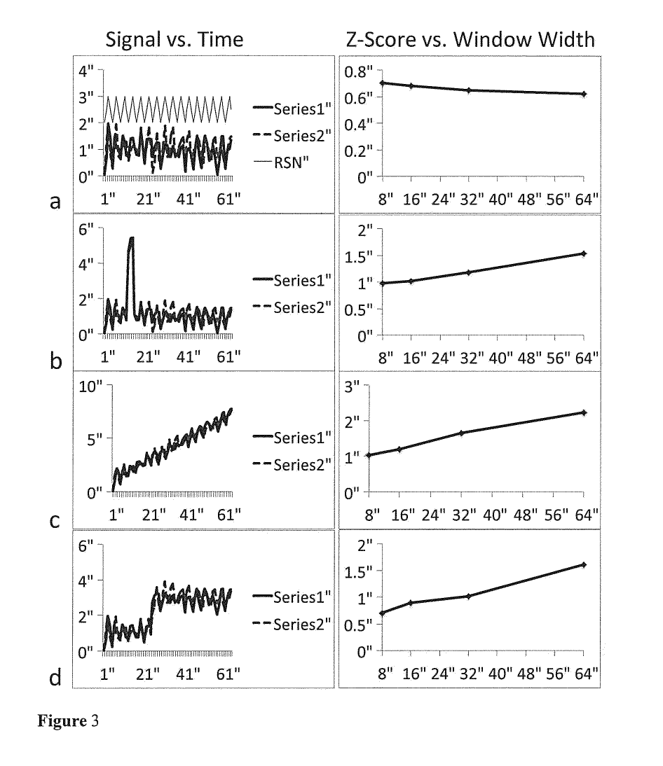

This seed-based connectivity approach has the following characteristics: The sliding window width K must be chosen to permit sampling of the lowest resting state frequency band at the Nyquist rate. For typical resting state frequencies between 0.025 and 0.3 Hz a sliding window width of approximately 10-20 s will be adequate. Shorter windows can be used to high-pass filter the resting state fluctuations and to further reduce the effect of confounding signal changes as shown in the simulations in FIGS. 3 to 5. However, decreasing the sliding window width below 10 s is expected to reduce the contrast to noise ratio of the Z-maps as the power spectrum amplitude of the resting state connectivity decreases rapidly with frequency. Of note, using short sliding windows for the correlation analysis does not reduce the frequency selectivity of the cumulative meta-statistics approach, since it is computed across the entire time series. In case of the sliding window meta-statistics approach the frequency selectivity is determined by the width of the meta-statistics sliding window. In addition to the running mean and the running standard deviation, the meta-statistics may include higher order statistics, such as kurtosis and skewness. Finally, as our preliminary data show, the meta-statistics approach may be performed using correlation maps instead of Fisher-Z transformed correlation maps.

FIGS. 3 to 5 simulate the application of the sliding window method with meta-statistics to resting state data that are confounded by signals of no interest.

FIG. 3 shows a simulation of the impact of spatially uniform confounding signal changes on resting state connectivity. The two signal time courses across 64 time points in FIG. 3a were generated by adding random numbers that were evenly distributed between 0 and 1 to an oscillatory signal waveform with amplitude 1 and periodicity of 4 time points. The corresponding mean Z-scores after segmenting the data using consecutive windows of 64, 32, 16 and 8 time points are shown in FIG. 3e. The dependence on the window width is minor. Adding a signal spike to both signal time courses (FIG. 3b) increases the mean Z-score. Adding a signal ramp to both signal time courses (FIG. 3c) increases the mean Z-score. Adding a time dependent signal offset to both signal time courses (FIG. 3d) increases the mean Z-score. The elevated Z-scores decrease with increasing degree of data segmentation (FIG. 3e-h)

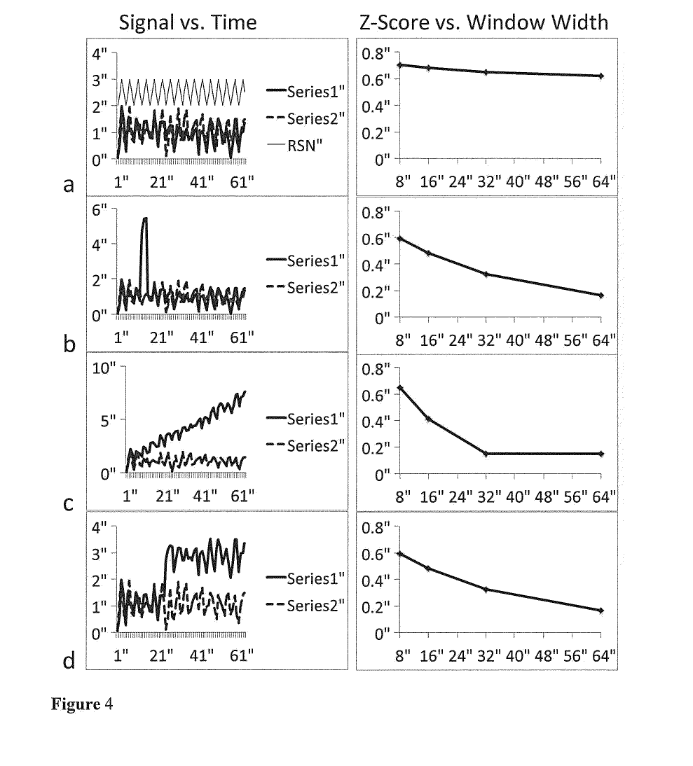

FIG. 4 shows a simulation of the impact of spatially nonuniform confounding signal changes on resting state connectivity. The two signal time courses across 64 time points in FIG. 4a were generated by adding random numbers that were evenly distributed between 0 and 1 to an oscillatory signal waveform with amplitude 1 and periodicity of 4 time points. The corresponding mean Z-scores after segmenting the data using consecutive windows of 64, 32, 16 and 8 time points are shown in FIG. 4e. The dependence on the window width is minor. Adding a signal spike to one of the two signal time courses (FIG. 4b) decreases the mean Z-score. Adding a signal ramp to one of the two signal time courses (FIG. 4c) decreases the mean Z-score. Adding a time dependent signal offset to one of the two signal time courses (FIG. 4d) decreases the mean Z-score. The decreased Z-scores increase with increasing degree of data segmentation almost to the level of the original signals without confounds (FIG. 3e-h)

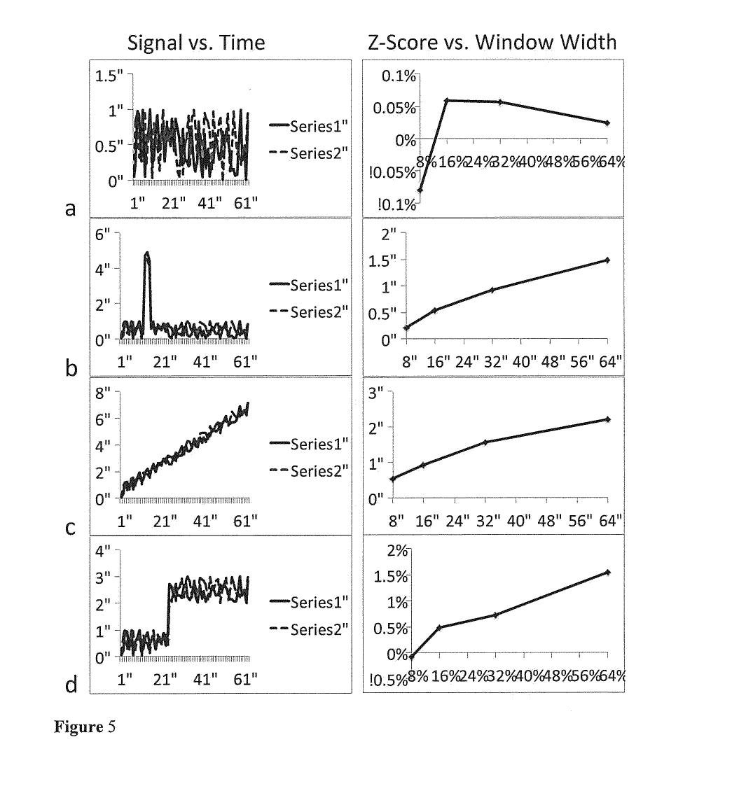

FIG. 5 shows a simulation of the impact of spatially uniform confounding signal changes on random noise signals. The two signal time courses across 64 time points in FIG. 5a were generated using random numbers that were evenly distributed between 0 and 1. The corresponding mean Z-scores after segmenting the data using consecutive windows of 64, 32, 16 and 8 time points are shown in FIG. 5e. The dependence on the window width is minor. Adding a signal spike to both signal time courses (FIG. 5b) increases the mean Z-score. Adding a signal ramp to both signal time courses (FIG. 5c) increases the mean Z-score. Adding a time dependent signal offset to both signal time courses (FIG. 5d) increases the mean Z-score. The increased Z-scores decrease with increasing degree of data segmentation almost to the level of the original signals without confounds (FIG. 5e-h)

In vivo validation of the sliding window method with meta-statistics

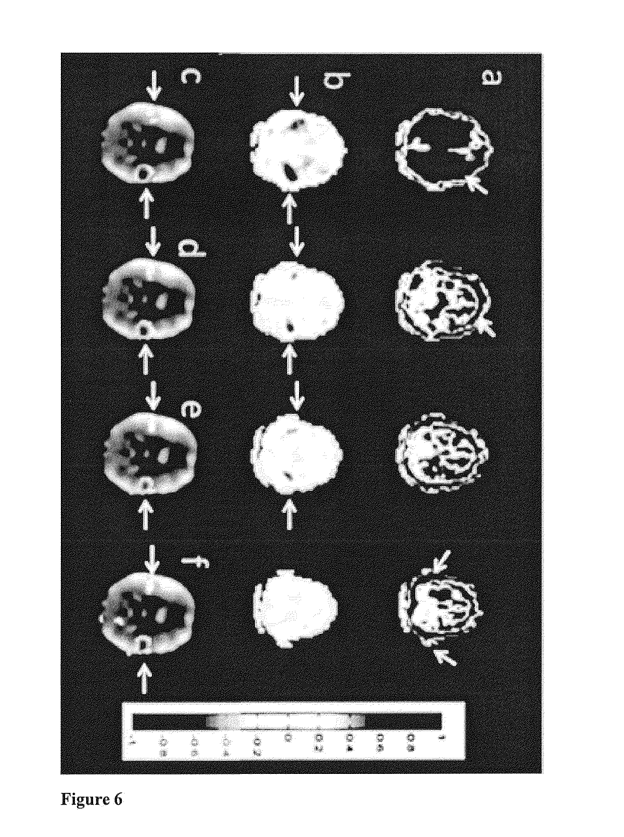

In preliminary studies using sensitive ultra-high-speed fMRI⁴⁵, and a custom designed real-time analysis software platform, we have shown feasibility of real-time mapping of seed-based connectivity (SBC) in healthy controls and patients with brain tumors, epilepsy and arteriovenous malformations. The data shown in FIG. 6 demonstrate the performance of the new approach using 2-slab echo-volumar imaging⁴⁵ with 4×4×6 mm³ voxel size, 14 acquired slices, TR: 136 ms and 2200 total volumes in a healthy control. Data were preprocessed with rigid body motion correction, a spatial 8×8×8 mm³ Gaussian filter and a 8 s moving average time domain filter for the cumulative correlation across the entire scan and the 15 s sliding window correlation. A 4 s filter was used for the 4 s sliding window correlation. A seed voxel seed was placed in motor cortex (Brodmann area 1 and 2) to measure resting state connectivity in the sensorimotor network. The initial 50 scans were discarded (N_{d}). Meta-statistics were computed at each TR starting at (N_{d}+N_{W}) and the final meta-statistics maps were used for individual subject analysis. Of note, the conversion of the correlation coefficients to Z-scores may be omitted, like in the present study, if the maximum correlation coefficient is on the order of 0.5 or less, since for this range the Fisher Z-transform is approximately the identity function. This is corroborated by our preliminary data shown in FIG. 6b. Cumulative correlation analysis across the entire scan without regression of confounding signal changes shows widely distributed coherent signal changes across the entire brain unrelated to resting state connectivity and edge artifacts due to movement (FIG. 6a). The cumulative meta-statistics mean across the sliding window correlation maps provided strong rejection of confounding signals from head movement, respiration, cardiac pulsation and signal drifts (FIG. 6b), without using regression of movement parameters and signals from white matter and CSF, and reveals the expected localization of the sensorimotor network in the mean meta-statistics map. The degree of rejection of confounding signals increased with decreasing sliding window width, while mean correlation coefficients decreased only slightly. A window width of 60 s often provided considerable artifact suppression, but a 15 s window was preferred due to even more robust artifact suppression. The correlation coefficients in white matter and CSF using this cumulative meta-mean across sliding window correlation maps were small, typically less than 0.2. Weighted subtraction of signals from white matter, CSF and the entire brain did not result in consistent improvement of mapping the major RSNs. Our preliminary data using this approach show that resting state connectivity exhibits correlation at time scales as short as 1 s. Seed-based connectivity of the auditory resting state network shown as mean meta-statistics across the sliding window correlation maps in this 5 min scan using sliding window widths of (FIG. 6c) 15 s, (FIG. 6d) 4 s, (FIG. 6e) 2 s and (FIG. 6f) 1 s demonstrates bilateral connectivity in the auditory cortex even at the shortest sliding window width of 1 s.

While the invention is suitable for mapping intrinsic signal variation in resting state fMRI it is also applicable for mapping functional networks and their connectivity in task-based fMRI, if at least one voxel location with task-related signal changes is known. This implementation is identical to the steps described above in [0029] to [0034] with the exception that the seed region corresponds to a task-activated brain region.

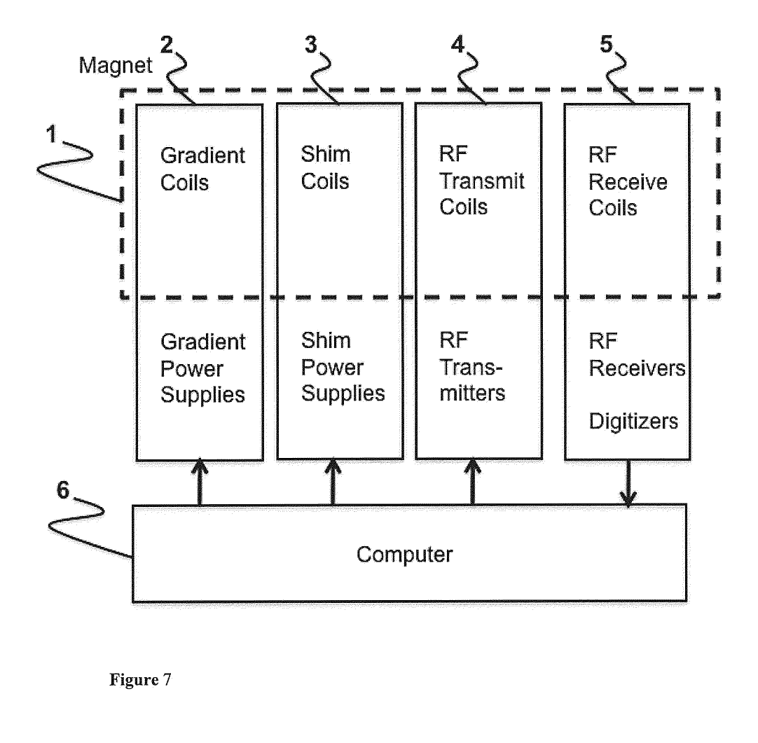

This fMRI methodology is implemented using a conventional MRI apparatus depicted in FIG. 7 for data collection. Briefly, the apparatus consists of a magnet 1 to generate a static magnetic field B₀, gradient coils and power supplies 2 to generate linear magnetic field gradients along the X, Y and Z axes, shim coils and shim power supplies 3 to generate higher order magnetic field gradients, single or multiple radiofrequency (RF) transmit coils and RF transmitter 4 to generate an RF field, single or multiple RF receiver coils forming an array, RF receivers and digitizers 5 to measure the received RF field, and a computer 6 to generate the pulse sequence, to measure and reconstruct the MR signals, to control the components of the MRI apparatus, and to analyze the reconstructed images. The computer performs real-time-data image reconstruction, preprocessing and the seed-based connectivity analysis described in this invention.

This combination of sliding window correlation analysis with the meta-statistics approach is applicable to other signal acquisition systems (e.g. magnetic resonance imaging (MRI) and spectroscopy (MRS), parallel MRI using array RF coils, electroencephalography, magneto-encephalography, optical imaging, recordings from electrode arrays, phased array radar, and radio-telescope arrays) where correlation between signals from different signal sources are examined in the presence of confounding signals of no interest.

What is claimed is:

1. A method for the evaluation of resting state functional MRI (fMRI) data from nuclear magnetic resonance tomographs that measures the correlation between a seed region signal time series and the signal time series in a plurality of voxels in the fMRI data comprising the steps of
performing fMRI measurements to create a series of fMRI data with N time points using a sampling interval Δt that is equal to or shorter than the Nyquist sampling interval 1/(2f) required for sampling a periodic resting state signal with frequency f, wherein f is the lowest frequency of interest in the resting state signal spectrum;
preprocessing of fMRI data using the steps of motion correction, slice time correction, spatial normalization into the space of a standardized brain atlas, spatial smoothing and time domain low pass filtering;
extraction of the signal time course in a seed region;
computation of the sliding window correlation between the signal time courses in said seed region and in a plurality of voxels in said fMRI data, utilizing K<N data values in said fMRI data series, in which, with continuing data measurement, the respective oldest values are discarded and the newest data values are employed in the computation, resulting in a series of sliding window correlation maps;
computation of the Fisher Z-transform of said series of sliding window correlation maps; and
computation of cumulative meta-statistics, including but not limited to the running mean and the running standard deviation across said series of Fisher Z-transformed correlation maps, and combinations thereof.

2. A method for the evaluation of fMRI data according to claim 1, further comprising the step of decreasing the sliding window width K to decrease the effect of signals of no interest on the meta-statistics, wherein said signals of no interest include, but are not limited to:
signal changes due to movement;
signal spikes; and
signal drifts.

3. A method for the evaluation of fMRI data according to claim 1, further comprising the step of selecting a minimum sliding window width K being equal to 1/(2fΔt), wherein f is the lowest frequency of interest in the resting state signal spectrum.

4. A method for the evaluation of fMRI data according to claim 1, further comprising:
computation of sliding window meta-statistics with window width L across a range of recently computed Z-maps Z(r, tᵢ), wherein K+L−1<N is the desired temporal resolution for monitoring changes in Z-scores during the scan and i=n−L, n−L+1, . . . , n−1, n. This sliding window meta-statistics includes but is not limited to the running sliding window mean and the running sliding window standard deviation across said series of Fisher Z-transformed correlation maps, and combinations thereof, in which, with continuing data measurement, the respective oldest values are discarded and the newest data values are employed in the computation of the meta-statistics maps.

5. A method for the evaluation of fMRI data according to claim 4, further comprising the computation of cumulative meta-statistics across said series of sliding window meta-statistics maps, including, but limited to the running mean and the running standard deviation, and combinations thereof.

6. A method for the evaluation of fMRI data according to claim 1, further comprising:
measurement of the rigid body movement parameters and their temporal derivatives in the K data points comprised in each of the sliding windows;
measurement of signals of no interest in selected regions of interest in the K data points comprised in each of the sliding windows, wherein said signals of no interest include, but are not limited to signal changes due to movement, signal spikes and signal drifts;
computation of the weights for each of said Fisher Z-transformed correlation maps, wherein said weights decrease with increasing amplitude of said rigid body movement parameters and their temporal derivatives, and increase with decreasing amplitude of said rigid body movement parameters and their temporal derivatives;
computation of the weights for each of said Fisher Z-transformed correlation maps, wherein said weights decrease with increasing amplitude of said signals of no interest and increase with decreasing amplitude of said signals of no interest;
computation of the product of the Fisher Z-transformed correlation maps and said weights for each sliding window position; and
computation of cumulative meta-statistics across said series of products, including, but not limited to the running mean and the running standard deviation, and combinations thereof.

7. A method for the evaluation of fMRI data according to claim 1, further comprising:
measurement of the rigid body movement parameters and their temporal derivatives in the K data points comprised in each of the sliding windows;
measurement of signals of no interest in selected regions of interest in the K data points comprised in each of the sliding windows, wherein said signals of no interest include, but are not limited to signal changes due to movement, signal spikes and signal drifts;
computation of the weights w(t), a confidence metric of Z(r, tₙ), which decreases when increasing levels of said signals of no interest are detected in the data measured within the sliding window K, and increases when said signals of no interest diminish. A preferred implementation uses the 6 measured translation and rotation parameters Δr(t), their temporal derivatives and the temporal derivative of the signal from a reference region δs(t) according to:
w(t)=1/(1−(α₁∫_{t-Δ}^{t}Δr(τ)dτ+α₂∫_{t-Δ}^{t}|δ(Δr(τ)/dτ|dτ−α₃∫_{t-Δ}^{t}|δs(τ)/dτ|dτ)), where
the scale factors aᵢ are determined experimentally;
utilization of polynomial functions of the arguments of the integrals;
utilization of thresholds for applying weights based on the movement parameters; and
computation of M_{w}(r, tₙ) the weighted cumulative meta-statistics across the series of Z-maps Z(r, tₙ), which include, but are not limited to the running mean M(mean, r, tₙ) and the running standard deviation M(SD, r, tᵢ) of Z(r, tₙ)*w(tₙ), and combinations thereof.

8. A method for the evaluation of fMRI data according to claim 1, further comprising:
measurement of the rigid body movement parameters and their temporal derivatives in the n data points comprised in each of the sliding windows;
measurement of signals of no interest in selected regions of interest in the K data points comprised in each of the sliding windows, wherein said signals of no interest include, but are not limited to signal changes due to movement, signal spikes and signal drifts; and
detrending of seed and target region signal time courses utilizing said rigid body movement parameters and their temporal derivatives, and of said signals of no interest.

9. A method for the evaluation of fMRI data according to claim 1, further comprising the application of thresholds to said running mean maps, running standard deviation maps and Fisher Z-transformed correlation maps using either of them. Examples include, but are not limited to:
a. Mapping the running means M(mean, r, tₙ) that are either less or greater than a threshold;
b. Mapping the running means M(mean, r, tₙ) whose running standard deviations M(SD, r, tₙ) is either less or greater than a threshold;
c. Mapping the running standard deviations M(SD, r, tₙ) either less or greater than a threshold;
d. Mapping the running standard deviations M(SD, r, tₙ) whose running mean M(mean, r, tₙ) is either less or greater than a threshold;
e. Mapping the ratios of the running means M(mean, r, tₙ) over the running standard deviations M(SD, r, tₙ) whose correlation is either less or greater than a threshold;
f. Mapping the ratios of the running standard deviations M(SD, r, tₙ) over the running means M(mean, r, tₙ) whose correlation is either less or greater than a threshold; and
g. Mapping the correlation R(r, tₙ) or the Z-scores Z(r,tₙ) whose running mean M(mean, r, tₙ) is either less or greater than a threshold.

10. A method for the evaluation of fMRI data according to claim 4, further comprising the application of thresholds to said sliding window mean maps, sliding window standard deviation maps and Fisher Z-transformed correlation maps using either of them. Examples include, but are not limited to:
a. Mapping the sliding window running means M(L, mean, r, tₙ) that are either less or greater than a threshold;
b. Mapping the sliding window running means M(L, mean, r, tₙ) whose sliding window running standard deviations M(L, SD, r, tₙ) is either less or greater than a threshold;
c. Mapping the sliding window running standard deviations M(L, SD, r, tₙ) either less or greater than a threshold;
d. Mapping the sliding window running standard deviations M(L, SD, r, tₙ) whose sliding window running mean M(L, mean, r, tₙ) is either less or greater than a threshold;
e. Mapping the ratios of the sliding window running means M(L, mean, r, tₙ) over the sliding window running standard deviations M(L, SD, r, tₙ) whose correlation is either less or greater than a threshold;
f. Mapping the ratios of the sliding window running standard deviations M(L, SD, r, tₙ) over the sliding window running means M(L, mean, r, tₙ) whose correlation is either less or greater than a threshold; and
g. Mapping the correlation R(r, tₙ) or the Z-scores Z(r,tₙ) whose sliding window running mean M(L, mean, r, tₙ) is either less or greater than a threshold.

11. A method for the evaluation of fMRI data according to claim 5, further comprising the application of thresholds to said running mean maps, running standard deviation maps and Fisher Z-transformed correlation maps using either of them. Examples include, but are not limited to:
a. Mapping the running means M(mean, r, tₙ) that are either less or greater than a threshold;
b. Mapping the running means M(mean, r, tₙ) whose running standard deviations M(SD, r, tₙ) is either less or greater than a threshold;
c. Mapping the running standard deviations M(SD, r, tₙ) either less or greater than a threshold;
d. Mapping the running standard deviations M(SD, r, tₙ) whose running mean M(mean, r, tₙ) is either less or greater than a threshold;
e. Mapping the ratios of the running means M(mean, r, tₙ) over the running standard deviations M(SD, r, tₙ) whose correlation is either less or greater than a threshold;
f. Mapping the ratios of the running standard deviations M(SD, r, tₙ) over the running means M(mean, r, tₙ) whose correlation is either less or greater than a threshold; and
g. Mapping the correlation R(r, tₙ) or the Z-scores Z(r,tₙ) whose running mean M(mean, r, tₙ) is either less or greater than a threshold.

12. A method for the evaluation of fMRI data according to claim 1, further comprising the steps of:
measuring task-induced signal changes during the execution of tasks including, but not limited to sensorimotor tasks, cognitive tasks, and mood induction tasks;
measuring a signal time course from a brain region that is known to be activated by the task;
selecting a sliding window width K being equal to or longer than 1/(2fΔt), wherein f is the lowest frequency in the power spectrum of the task activation paradigm; and
computation of the sliding window correlation between the signal time courses in said seed region and in a plurality of voxels in said fMRI data, utilizing said number of K data values in said fMRI data series, in which, with continuing data measurement, the respective oldest values are discarded and the newest data values are employed in the computation, resulting in a series of correlation maps;
computation of the Fisher Z-transform of said series of sliding window correlation maps; and
computation of cumulative meta-statistics, including but not limited to the running mean and the running standard deviation across said series of Fisher Z-transformed correlation maps, and combinations thereof.

13. A method for the evaluation of functional MRI (fMRI) data according to claim 1, further comprising the steps of additionally measuring higher order meta-statistics, including, but not limited to kurtosis and skewness.

14. A nuclear magnetic resonance tomograph for mapping connectivity and function in the brain including a computer for the evaluation of data from the nuclear magnetic resonance tomograph comprising:
an RF pulse transmitting device to excite nuclear spins in a circumscribed region;
a gradient pulse application device to localize signals and encode k-space;
a pulse sequence control device that generates an fMRI pulse sequence;
an NMR signal receiving device that collects a series of fMRI raw data with N time points using a sampling interval At that is equal to or shorter than the Nyquist sampling interval 1/(2f) required for sampling a periodic resting state signal with frequency f, wherein f is the lowest frequency of interest in the resting state signal spectrum;
a data collection, reconstruction and storage device that generates a series of fMRI images; and
a real-time data analysis device that performs the steps of fMRI preprocessing, extraction of a plurality of seed signal time courses, computation of the sliding window correlation between the signal time courses in said seed region and in a plurality of voxels in said fMRI data, utilizing K<N data values in said fMRI data series, in which, with continuing data measurement, the respective oldest values are discarded and the newest data values are employed in the computation, resulting in a series of correlation maps, computation of the Fisher Z-transform of said series of sliding window correlation maps, and computation of cumulative meta-statistics, including but not limited to the running mean and the running standard deviation across said series of Fisher Z-transformed correlation maps, and combinations thereof.

15. A functional magnetic resonance imaging apparatus according to claim 14, further comprising the step of decreasing the sliding window width K to decrease the effect of signals of no interest on the meta-statistics, wherein said signals of no interest include, but are not limited to:
signal changes due to movement;
signal spikes; and
signal drifts.

16. A functional magnetic resonance imaging apparatus according to claim 14, further comprising the step of selecting a sliding window width K being equal to 1/(2fΔt), wherein f is the lowest frequency of interest in the resting state signal fluctuation.

17. A functional magnetic resonance imaging apparatus according to claim 14, further comprising:
computation of sliding window meta-statistics with window width L across a range of recently computed Z-maps Z(r, tᵢ), where K+L−1<N is the desired temporal resolution for monitoring changes in Z-scores and i=n−L, n−L+1, . . . , n−1, n. This sliding window meta-statistics includes, but is not limited to the running sliding window mean and the running sliding window standard deviation across said series of Fisher Z-transformed correlation maps, and combinations thereof, in which, with continuing data measurement, the respective oldest values are discarded and the newest data values are employed in the computation.

18. A functional magnetic resonance imaging apparatus according to claim 14, further comprising:
measurement of the rigid body movement parameters and their temporal derivatives in the K data points comprised in each of the sliding windows;
measurement of signals of no interest in selected regions of interest in the K data points comprised in each of the sliding windows, wherein said signals of no interest include, but are not limited to signal changes due to movement, signal spikes and signal drifts;
computation of the weights for each of said Fisher Z-transformed correlation maps, wherein said weights decrease with increasing amplitude of said rigid body movement parameters and their temporal derivatives, and increase with decreasing amplitude of said rigid body movement parameters and their temporal derivatives;
computation of the weights for each of said Fisher Z-transformed correlation maps, wherein said weights decrease with increasing amplitude of said signals of no interest and increase with decreasing amplitude of said signals of no interest;
computation of the product of the Fisher Z-transformed correlation maps and said weights for each sliding window position; and
computation of cumulative meta-statistics across said series of products, including, but not limited to the running mean and the running standard deviation, and combinations thereof.

19. A signal processing apparatus that is applicable to signal acquisition systems including, but not limited to magnetic resonance imaging (MRI) and spectroscopy (MRS), parallel MRI using array RF coils, electroencephalography, magneto-encephalography, optical imaging, recordings from electrode arrays, phased array radar, and radio-telescope arrays, wherein correlation between signals from different signal sources is examined in the presence of confounding signals of no interest, comprising the steps of:
performing measurements to create a plurality of source data series with N time points using a sampling interval Δt that is equal to or shorter than the sampling interval 1/(2f) required for sampling a periodic resting state signal with frequency f at the Nyquist rate, wherein f is the lowest frequency of interest in the signal spectrum;
preprocessing of said plurality of source data series using preprocessing steps that are customary for the acquisition method in use, but excluding the regression of signals of no interest;
extraction of a reference signal time course from said plurality of source data series;
computation of the sliding window correlation between said reference signal time course and said plurality of source data series, utilizing K<N data values in said source data series, in which, with continuing data measurement, the respective oldest values are discarded and the newest data values are employed in the computation, resulting in a series of correlation maps;
computation of the Fisher Z-transform of said series of sliding window correlation maps; and
computation of cumulative meta-statistics, including but not limited to the running mean and the running standard deviation across said series of Fisher Z-transformed correlation maps, and combinations thereof.

20. A signal processing apparatus according to claim 19, further comprising the step of decreasing the sliding window width K to decrease the effect of signals of no interest on the meta-statistics, wherein said signals of no interest include, but are not limited to:
signal changes due to movement;
signal spikes; and
signal drifts.
